(19) United States
(12) Patent Application Publication
(10) Pub. No.: US 2012/0004559 A1
(43) Pub. Date: Jan. 5, 2012

(54) Blood Pressure Meter

(75) Inventors: WEN LING LEE, TAOYUAN COUNTY (TW); CHIH MING CHEN, NEW TAIPEI CITY (TW)

(73) Assignee: AVITA CORPORATION, NEW TAIPEI CITY (TW)

(21) Appl. No.: 13/175,815

(22) Filed: Jul. 1, 2011

(30) Foreign Application Priority Data
Jul. 2, 2010 (TW) 099212682
Sep. 26, 2010 (CN) 201020541858.6

(51) Int. Cl. A61B 5/022

(52) U.S. Cl. 600/499

(57) ABSTRACT

A blood pressure meter includes a cuff unit and a clip unit. The cuff unit includes at least one first connecting portion and at least one second connecting portion. The clip unit is detachably connected with the cuff unit and comprises a first clipping portion for each first connecting portion and a second clipping portion for each second connecting portion, wherein the first and the second clipping portions are pivotally connected in such a manner that the first clipping portion is connected to the first connecting portion in a detachable manner and the second clipping portion is connected to the second connecting portion in a detachable manner, thereby the blood pressure meter is advantageous in that accurate positioning and operation for blood pressure monitoring is achieved.

### BACKGROUND OF THE PRESENT INVENTION

1. Field of Invention

The present invention relates to a blood pressure meter, and more particular to a blood pressure meter which includes a cuff unit which is capable of guiding a positioning and an operation of the cuff unit for blood pressure monitoring.

2. Description of Related Arts

Nowadays, self-monitoring system for health control becomes more and more common when level of general knowledge increases with technological advancement. People starts to emphasize on self-measuring or self-monitoring so as to grasp more understanding about his or her health conditions, which is one of the crucial steps for health control.

As shown in FIG. 1 of the drawings, a conventional blood pressure meter 1 includes a main body 11, a cuff 12 and a connecting duct 13. When measuring the blood pressure of a user, the cuff 12 is put onto an upper arm of the user to enclose the upper arm tightly which is then inflated by the main body 11 through the connecting duct 13 when the start button of the main body 11 is pressed. When the cuff is inflated to a certain level, the inflating gas of the cuff 12 starts to release. After a certain period of time, the blood pressure readings are obtained. The systolic and diastolic pressures as well as the pulse rate (beat per minute) is obtained and displayed on the main body 11.

However, there are some drawbacks for this type of conventional blood pressure meter in view of the measuring process. First, it is not easy for the user to put the cuff 12 on his or her upper arm. Second, the user may put the cuff 12 in an opposite direction and the direction of the connecting duct is being reversed, causing inaccurate measurement or unsmooth inflating process. On the other hands, in view of actual operation, the diameters of upper arms of different users are different and the cuff 12 may not fit all of the users.

Accordingly, a cuff, whish is easy to use and guidable to position as well as universally fit to all users, becomes an important issue for the blood pressure meter.

### SUMMARY OF THE PRESENT INVENTION

The invention is advantageous in that it provides a blood pressure meter which includes a cuff unit which is positioned for easy handling and operation while capable of being guided into position to ensure accurate positioning and measurement, while having a size which is universally fit for different users for use at different locations of a body of the user.

Additional advantages and features of the invention will become apparent from the description which follows, and may be realized by means of the instrumentalities and combinations particular point out in the appended claims.

According to the present invention, the foregoing and other objects and advantages are attained by a blood pressure meter, comprising a cuff unit and a clip unit. The cuff unit comprises at least one first connecting portion and at least one second connecting portion provided thereon. The clip unit is detachably connected with the cuff unit, comprising a first clipping portion and a second clipping portion pivotally connected with the first clipping portion, wherein the first clipping portion is arranged for connecting to the corresponding first connecting portion in a detachable manner and the second clipping portion is arranged for connecting to the corresponding second connecting portion in a detachable manner.

Preferably, the blood pressure meter further comprises a positioning unit which comprises a first position member provided on the cuff unit; and a second position member provided on the cuff unit or the clip unit, arranged for coupling with the first position member in such a manner that the cuff unit is secured into position.

Preferably, the positioning unit comprises a positioning mechanism through which the first position member and the second position member are connected, wherein the positioning mechanism is selected from a group consisting of adhesive mechanism, hook and loop fastening mechanism, clicking mechanism, and button-type mechanism.

Preferably, the blood pressure meter further comprises a resilient member provided at a hinge portion of the clip unit.

Preferably, the resilient member is a torsion spring.

Preferably, the blood pressure meter further comprises a connecting mechanism through which the first clipping portion and the second clipping portion are connected to the first connecting portion and the second connecting portion respectively, wherein the connecting mechanism is selected from the group consisting of coupling mechanism, hooking mechanism, bonding mechanism, clicking mechanism and buckling mechanism.

Preferably, the clip unit further comprises a first adjustment member and a second adjustment member complementary and connected to the first clipping portion and the second clipping portion respectively.

Preferably, the first adjustment member and the second adjustment member comprise at least one first locking member provided thereon.

Preferably, the blood pressure meter includes a second locking member corresponding to one the first locking member which is provided on the first clipping portion and the second clipping portion.

Preferably, the first locking member or the second locking member has a protruded structure which is a protruded teeth structure, a protruded bump structure, a protruded block structure or a protruded bar structure.

Preferably, each of the first clipping portion and the second clipping portion has at least one hook member, wherein the hook members of the first clipping portion and the second clipping portion are arranged for hooking with the first connecting portion and the second connecting portion respectively such that the first clipping portion and the second clipping portion are locked into position with respective to the first connecting portion and the second connecting portion respectively.

As mentioned above, the clip unit is detachably connected to the cuff unit, wherein the first and the second clipping portions are pivotally connected. Accordingly, the cuff unit can be open and placed in a specific location of a body of a user through pressing the clipping unit to an open condition and secured into position through releasing the pressing force on the clipping unit to a closed condition. In addition, a plurality of clip units of different sizes can be provided for providing a plurality of sizes for the cuff unit to fit different user, or adjustment members are provided in the clipping portions for length adjustment of the cuff unit. The blood pressure meter may further include a positioning unit which comprises a first and a second position members for securing the cuff unit into position in such a manner that the cuff unit will not be loosened to open during the inflation process. Accordingly, the cuff unit is guided to secure into position on a user and can be stabilized for operation during the inflation process, thereby a user can position and operate the cuff unit easily. In addition, since the user can make use of the clip unit to hold and position the cuff unit, the cuff unit being misplaced and misused can be avoided, thereby avoiding measuring errors or unsmooth inflation process. Furthermore, the clip unit is connected to the cuff unit in a detachable manner, thereby the clip unit can be replaced by another clip unit of different size to fit a size of the user, or adjustment members are provided for size adjustment purpose. Therefore, flexibility in size is provided.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is an illustration of a conventional blood pressure meter.

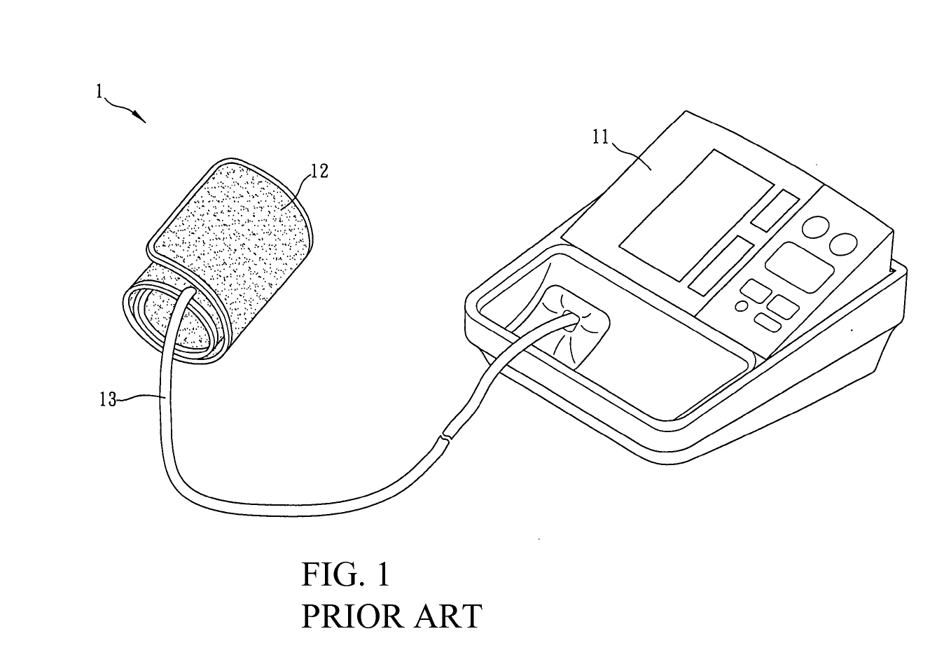

FIG. 2A is a partially exploded view illustration of a blood pressure meter according to a preferred embodiment of the present invention.

FIG. 2B is an illustration of a blood pressure meter according to the above preferred embodiment of the present invention.

FIG. 3 is a partially exploded view illustration of another exemplary embodiment of a blood pressure meter according to the above preferred embodiment of the present invention.

FIG. 4 is an illustration of another exemplary embodiment of a blood pressure meter according to the above preferred embodiment of the present invention.

FIG. 5 is an illustration of another exemplary embodiment of a blood pressure meter according to the above preferred embodiment of the present invention.

### DETAILED DESCRIPTION OF THE PREFERRED EMBODIMENT

The following description and the drawings are used to describe the blood pressure meter according to a preferred embodiment of the present invention, where the numerical references for identical elements are the same.

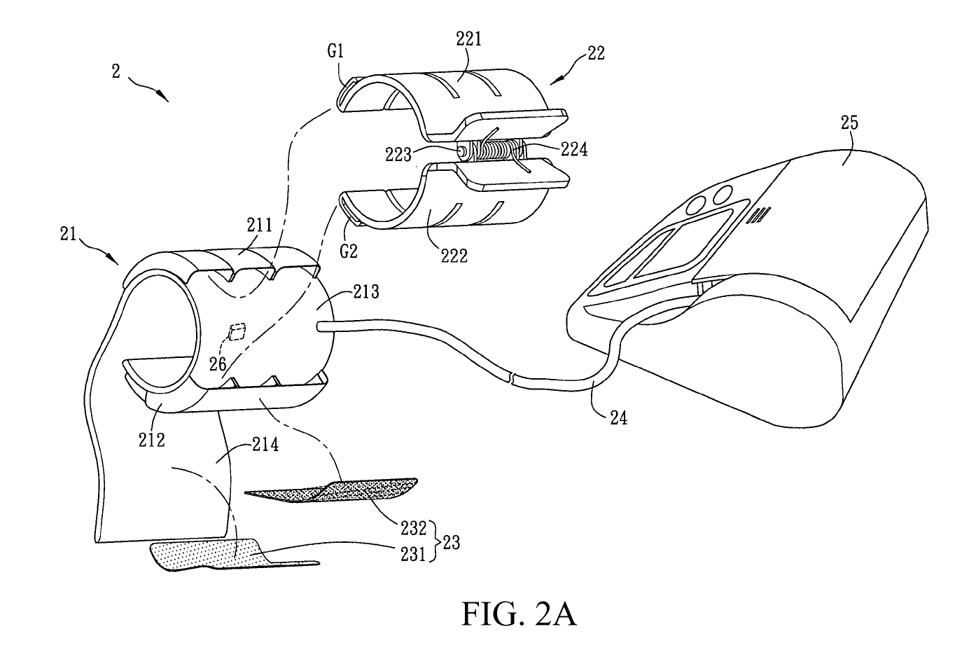

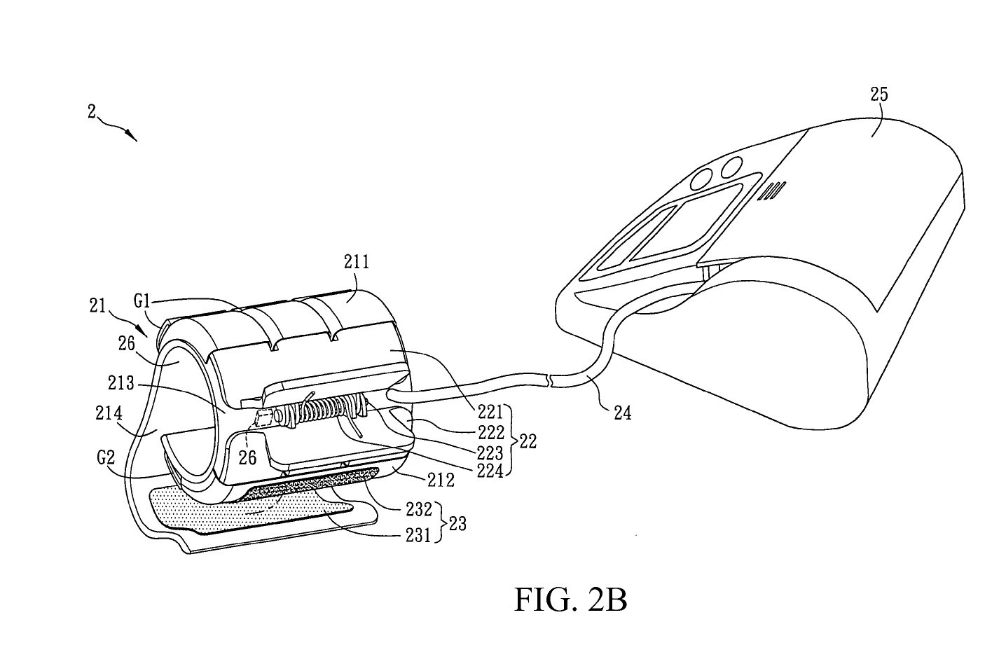

Referring to FIGS. 2A and 2B of the drawings, which are a partially exploded and a perspective illustration of the blood pressure meter 2 respectively according to a preferred embodiment of the present invention. The blood pressure meter 2 comprises a cuff unit 21 and a clip unit 22. In additional, the blood pressure meter 2 may further comprise a positioning unit 23. The blood pressure meter 2 of the present invention is capable of being used for blood pressure measuring through various parts of a human body. In other words, it is not necessarily to position the cuff unit 21 on the upper arm and it is possible to position the cuff unit 21 onto different locations of the human body such as the arm, the wrist, the ankle or the finger of a measuring object. The measuring location of the human body is not limited.

The cuff unit 21 comprises at least one first connecting portion 211 and at least one second connecting portion 212. As shown in FIGS. 2A and 2B of the drawings, one first connecting portion 211 and one second connecting portion 212 are illustrated as exemplary embodiment only. The first and the second connecting portion 211, 212 are positioned on a first side 213 of the cuff unit 21.

The clip unit 22 is detachably connected to the cuff unit 21 and comprises a first clipping portion 221 and a second clipping portion 222 for coupling to the first connecting portion 211 and the second connecting portion 212 respectively. In other words, the clip unit 22 is capable of being installed and connected to the cuff unit 21, or detached and removed from the cuff unit 21. The first clipping portion 221 and the second clipping portion 222 are connected through a clip portion connector 223. Preferably, the cuff unit 21 further comprises a resilient member 224 at a position that the first and the second clip units 221, 222 are connected therebetween. Preferably, the resilient member 224 is provided at a hinge portion of said clip unit 21. The resilient member 224 can be a torsion spring positioned between the first and the second clipping portion 221, 222 which is connected to the clip portion connector 223 in such a manner that the first and the second clipping portion 221, 222 are capable of providing a clipping force. In other words, the interconnection of first and the second clip units 221, 222 has clipping ability through the resilient member 224 for providing spring action. According to the preferred embodiment of the present invention, the clip unit 22 is a crocodile clip (spring clip) made of plasticity materials, resilient materials, metal or alloy materials such as plastic, rubber or memory alloy.

The first clipping portion 221 is connected with the first connecting portion 211 and the second clipping portion 222 is connected with the second connecting portion 212. The first and the second clipping portions 221, 222 are connected to the first and the second connecting portion 211, 212 respectively by a plurality of connecting mechanisms such as coupling, hooking, bonding, clicking or buckling mechanisms. According to the preferred embodiment of the present invention, the first and the second clipping portions 221, 222 are connected to the first and the second connecting portion 211, 212 respectively by coupling through penetrating through-holes provided on the first and the second clipping portions 221, 222 and the first and the second connecting portion 211, 212.

In particular, the first clipping portion 221 comprises at least one first hook member G1 such that the first clipping portion 221 is securely connected to the first connecting portion 211, while the second clipping portion 222 comprises at least one second hook member G2 such that the second clipping portion 222 is securely connected to the second connecting portion 212. The first and the second hook members G1, G2 are corresponding to the first and the second connecting portions 211, 212 respectively such that the first and the second clipping portions 221, 222 are detachably and securely connected to the first and the second connecting portions 221, 222 through the first and the second hook members G1, G2 respectively. Preferably, the first and the second clipping portion 221, 222 comprises three first hook member G1 and three second hook member G2 respectively. However, it is worth mentioning that the number of corresponding first and second hook members G1, G2 can be varied, and the number of first hook member G1 is not necessarily the same as the number of second hook member G2.

The positioning unit 23 comprises a first position member 231 and a second position member 232. The first position member 231 is provided on the cuff unit 21 while the second position member 232 is provided on the cuff unit 21 or the clip unit 22. As illustrated in the figures, the first position member 231 is provided on a second side 214 of the cuff unit 21 (which is the opposite side of the first side 213 of the cuff unit 21), and the second position member 232 is provided on the second connecting portion 212 of the cuff unit 21. It is worth mentioning that when the second clipping portion 222 is connected to the second connecting portion 212 through bonding, clicking or buckling, the second position member 232 can also be partially provided on the second clipping portion 222 of the clip unit 22.

The first position member 231 is complementary to the second position member 232. According to the preferred embodiment of the present invention, the first and the second position members 231, 232 are VELCRO fasteners (a type of hook and loop fastener) which are adhered together in an overlapped manner. However, the first and the second position members 231, 232 are not limited to hook and loop fasteners (VELCRO) and can be connected together through other connecting mechanisms such as other types of hook and loop fastening mechanism, clicking or coupling mechanisms. Accordingly, the cuff unit 21 is fittingly and securely positioned on the arm of a user through the complementary connection between the first and the second position members 231, 232, while the cuff unit 21 is counteractive to any expansion force and loosening effect during the inflating process and is maintained to lock into position. On the other hand, length of the cuff unit 21 is adjustable through re-positioning the first and the second position members 231, 232. Therefore, the measuring process can be carried out easily and smoothly.

Furthermore, the blood pressure meter 2 comprises a main body 25 and a duct unit 24 connecting between the cuff unit 21 and the main body 25 such that the cuff unit 21 is inflated by the main body 25 through the connecting duct unit 24 during blood pressure measuring. It is worth mentioning that the cuff unit 21 can be inflated by manual inflation or automated inflation mechanism.

The blood pressure meter 2 further comprises a sensor unit 26 provided on the cuff unit 21 or on the main body 25 which is arranged for electrically connecting to the main body 25. According to the preferred embodiment, the sensor unit 26 is provided on the cuff unit 21. The sensor unit 26 is capable of detecting a pressure change at micro level which is generated through resonance between the cuff unit 21 and the blood vessel of a user during the inflating process. The sensor unit 26 is arranged for converting the pressure change at micro level to electrical signals which is then transmitted to the main body 25. The main body 25, in response to the signals from the sensor unit 26 and through data calculation and conversion, is capable of providing an output information which includes systolic and diastolic pressures and a pulse rate of the user. Alternately, the sensor unit 26 can also be positioned inside the main body 25 for detecting the pressure change transmitted to the main body 25 through the connecting duct unit 24.

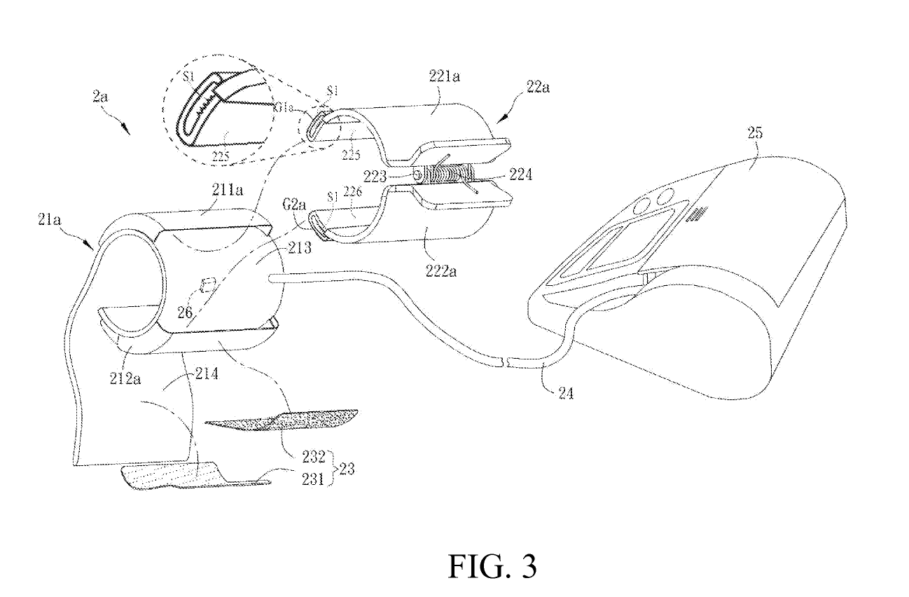

Referring to FIG. 3 of the drawings, another exemplary embodiment of the blood pressure meter 2a according to the preferred embodiment of the present invention is illustrated.

In order to fit different arm sizes or arm diameters, the exemplary embodiment of the blood pressure meter 2a as illustrated is different from the blood pressure meter 2 in that the clip unit 22a further comprises a first adjustment member 225 and a second adjustment member 226 complementary to the first clipping portion 221 and the second clipping portion 222 respectively such that the clip unit 22a is capable of providing a clipping range for a plurality of diameters to fit different arm sizes of different users. As shown in the FIG. 3 of the drawings, the first clipping portion 221a and the second clipping portion 222a comprises one first and second hook members respectively. It is worth mentioning that the first adjustment member 225 and the second adjustment member 226 couple with the first clipping portion 221a and the second clipping portion 222a respectively in such a manner that the first and the second clipping portions 222a, each having a groove portion, are arranged to receiving the first and the second adjustment portions 225, 226 respectively for locking the first and the second adjustment portions 225, 226 into position with respect to the first and the second clipping portions 221a, 222a respectively. Alternately, the first adjustment member 225 and the second adjustment member 226 couple with the first clipping portion 221a and the second clipping portion 222a respectively in such a manner that the first adjustment member 225 and the second adjustment member 226, each having a groove portion, are arranged to receiving the first and the second clipping portions 221a, 222a respectively for locking the first and the second adjustment portions 225, 226 into position with respect to the first and the second clipping portions 221a, 222a respectively.

Each of the first adjustment member 225 and the second adjustment member 226 comprises at least one first locking member S1 for connecting the first clipping portion 221a and the second clipping portion 222a to the first adjustment member 225 and the second adjustment member 226 respectively, while each of the first clipping portion 221a and the second clipping portion 222a comprises at least one second locking member (not shown in the figure) corresponding to the first locking member S1 respectively. Preferably, the first locking member S1 and the second locking member are constructed to having a protruded structure which includes a protruded teeth structure, a protruded bump structure, a protruded block structure and/or a protruded bar structure, which are provided on the adjustment member and the clipping portion in such a manner that the first locking member S1 and the second locking member are constructed to having a protruded structure which are complementary with each other. As shown in the figure, the first locking member S1 has a protruded teeth structure such that the first adjustment member 225 and the second adjustment member 226 is complementary to and connected to the first clipping portion 221a and the second clipping portion 222a.

In particular, at least one hook member is provided on the first adjustment member 225 and the second adjustment member 226 respectively which is arranged for hooking to the first connecting portion 211a and the second connecting portion 212a respectively. The clip unit 22a, which is different from the clip unit 22, comprises one first adjustment member 225 and one second adjustment member 226; and two hook members G1a, G2a for the first and the second adjustment members 225, 226 respectively, while the first and the second connecting portion 211a, 212a of the cuff unit 21a are constructed in such a manner that the first and the second connecting portion 211a, 212a have a structure which is complementary to and is arranged for coupling with the two hook members G1a, G2a for the first and the second adjustment members 225, 226 respectively, thereby the two hook members G1a, G2a for the first and the second adjustment members 225, 226 are capable of hooking onto the first and the second connecting portions 211a, 212a respectively.

Accordingly, since the blood pressure meter 2 comprises the clip unit 22 which is connected to the cuff unit 21 to which the first and the second clipping portions 221, 222 are connected, the cuff unit 21 can be open for fitting into a user for measuring through pressing the clip unit 22 and the cuff unit is secured into position through connecting the first and the second position members 231, 232 of the positioning unit 23, thereby the blood pressure meter 2 is ready for use. On the other hand, when the blood pressure meter 2 is used by a user with small arm size, such as a baby or a kid, a length of the clip unit 22 can be adjusted through replacing the clip unit 22 by the clip unit 22a, or through adjusting a length of the first and the second adjustment members 225, 226 to which the first and the second clipping portions 221a, 222a are complementarily connected such that an arm size of the cuff unit 21a is set through the clip unit 22a. Since a user of the blood pressure meter of the present invention can face towards the blood pressure meter or hold the clip unit 22 to setting the cuff unit 21, 21a into position easily, the problem of setting the cuff unit 21 in an incorrect direction or with the wrong side is solved and hence the measuring error is avoided. Furthermore, the clip unit 22 is detachably connected to the cuff unit 21, therefore the clip unit 22 can be detached and replaced with another clip unit 22 of different size for use with users having different arm sizes. In other words, the flexibilities in size is provided through the detachable clip unit 22 which is arranged to connecting to the cuff unit 21a for setting a size of the cuff unit 21a.

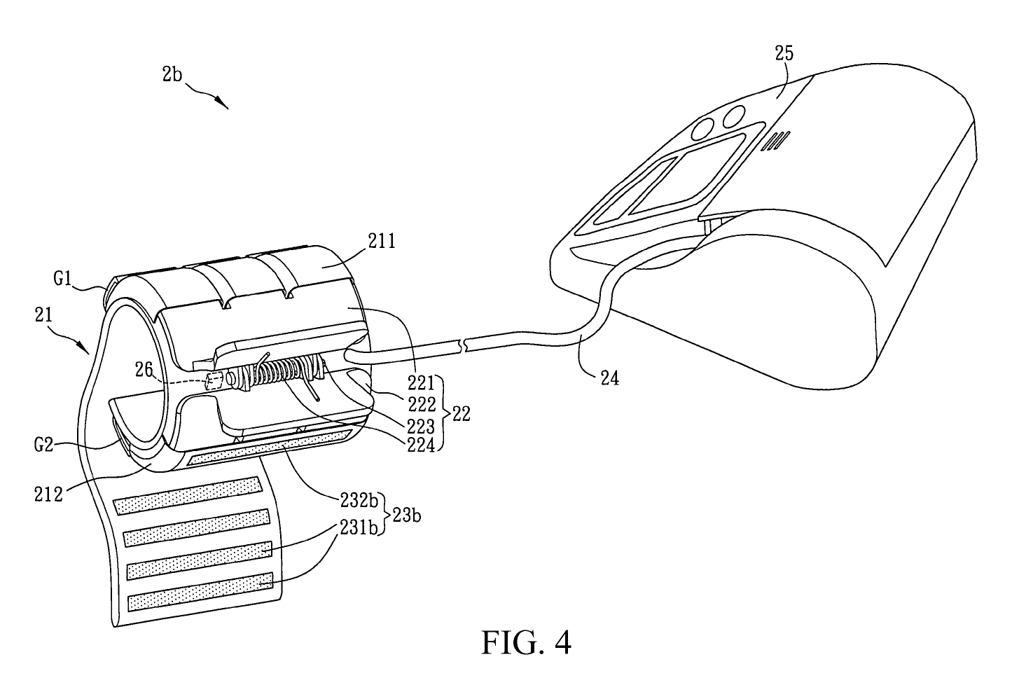

On the other hand, referring to FIG. 4 of the drawings, another exemplary embodiment of the blood pressure meter 2b is illustrated.

The blood pressure meter 2b is different from the blood pressure meter 2 in that the first and second position members 231b, 232b of the positioning unit 23b of the blood pressure meter 2b are a plurality set of magnetic fasteners (or hook and loop fasteners) in which the first and the second position members 231a, 232b are connected through a connecting mechanism which employs the attractive force of magnetism (or adhesive force of the hook and loop fastener).

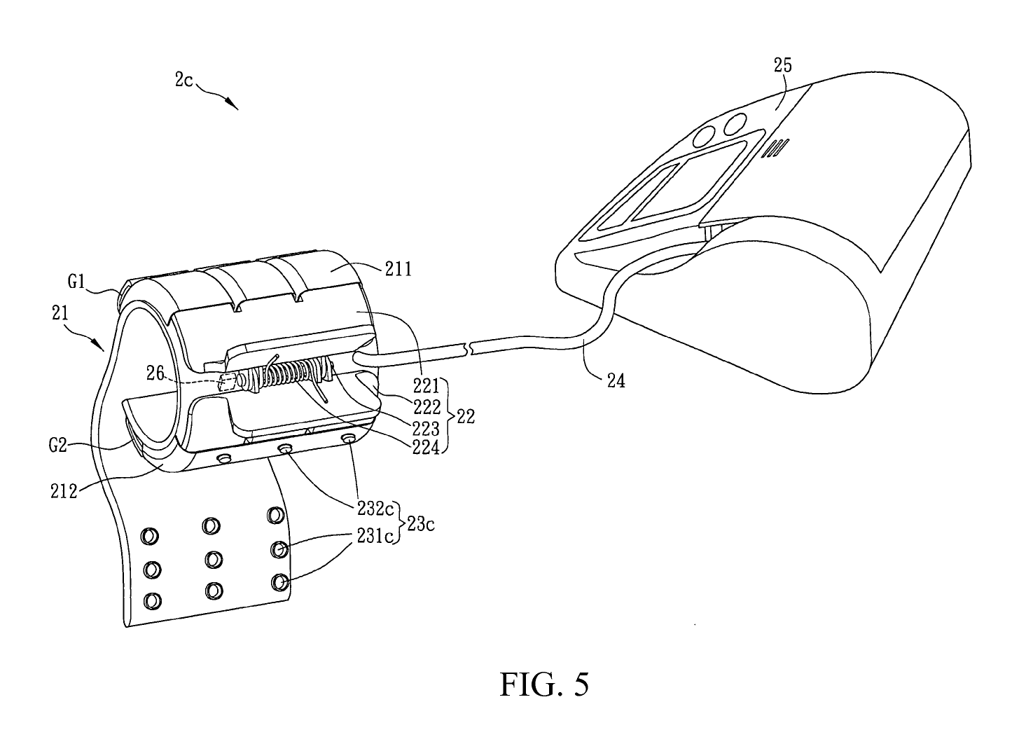

In addition, referring to FIG. 5 of the drawings, another exemplary embodiment of the blood pressure meter 2c is illustrated.

The blood pressure meter 2c is different from the blood pressure meter 2 in that the first and second position members 231b, 232b of the positioning unit 23b of the blood pressure meter 2b are a plurality set of button connectors in which the first and the second position members 231a, 232b are connected through a connecting mechanism, which is the coupling mechanism.

Other elements of the blood pressure meter 2a, 2b, 2c are referred to the description for the blood pressure meter 2 which is not repetitively described.

Accordingly, the blood pressure meter of the preferred embodiment of the present invention comprises a clip unit connecting to a cuff unit, wherein the clip unit comprises a first clipping portion and a second clipping portion complementarily and connecting to the first clipping portion, thereby the cuff unit is capable of being open to enclose and fit into a user for measuring through the clip unit while a plurality of clip units of different sizes can be selectively replaceable with each other for fitting different users, or through the adjustment members of the clip unit to adjust a diameter of the cuff unit for fitting different parts of body of the user for measuring. The blood pressure meter may further comprises a first and a second position members connecting with each other for securing the cuff unit into position such that the cuff unit will not expand and distort in position during the inflation process, thereby the cuff unit can be securely positioned at a preset location (such as upper arm) of a body for measuring. Accordingly, the user can easily position the cuff unit and secure the cuff unit into position easily and the problem of position displacement or blockage of gas supply to the cuff unit is solved. In addition, since the clip unit has to fit different users who require different cuff sizes, the clip unit is connected to the cuff unit in a detachable manner such that a length of the adjustment members of the corresponding clipping portions can be adjusted or the clip unit can be replaced with another clip unit of different size to fit different users, thereby flexibilities in size of the blood pressure meter is provided.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A blood pressure meter, comprising:
a cuff unit which comprises at least one first connecting portion and at least one second connecting portion provided thereon; and
a clip unit detachably connected with said cuff unit which comprises a first clipping portion for each said first connecting portion of said cuff unit; and a second clipping portion pivotally connected with said first clipping portion for each said second connecting portion of said cuff unit, wherein said first clipping portion is arranged for connecting to said corresponding first connecting portion in a detachable manner and said second clipping portion is arranged for connecting to said corresponding second connecting portion in a detachable manner.

2. The blood pressure meter, as recited in claim 1, further comprising a positioning unit which comprises a first position member provided on said cuff unit, and a second position member provided on said cuff unit or said clip unit, arranged for coupling with said first position member in such a manner that said cuff unit is secured into position.

3. The blood pressure meter, as recited in claim 2, wherein said positioning unit comprises a positioning mechanism through which said first position member and said second position member are connected, wherein said positioning mechanism is selected from the group consisting of adhesive mechanism, hook and loop fastening mechanism, clicking mechanism, and button-type mechanism.

4. The blood pressure meter, as recited in claim 1, further comprising a resilient member provided at a hinge portion of said clip unit.

5. The blood pressure meter, as recited in claim 4, wherein said resilient member is a torsion spring.

6. The blood pressure meter, as recited in claim 1, further comprising a connecting mechanism through which said first clipping portion and said second clipping portion are connected to said first connecting portion and said second connecting portion respectively, wherein said connecting mechanism is selected from the group consisting of coupling mechanism, hooking mechanism, bonding mechanism, clicking mechanism and buckling mechanism.

7. The blood pressure meter, as recited in claim 1, wherein said clip unit further comprises a first adjustment member and a second adjustment member complementary and connected to said first clipping portion and said second clipping portion respectively.

8. The blood pressure meter, as recited in claim 7, wherein said first adjustment member and said second adjustment member comprises at least one first locking member provided thereon.

9. The blood pressure meter, as recited in claim 8, further comprising a second locking member corresponding to one said first locking member which is provided on said first clipping portion and said second clipping portion.

10. The blood pressure meter, as recited in claim 8, wherein said first locking member has a protruded structure selected from a group consisting of a protruded teeth structure, a protruded bump structure, a protruded block structure, and a protruded bar structure.

11. The blood pressure meter, as recited in claim 9, wherein each of said first locking member and said second locking member has a protruded structure selected from a group consisting of a protruded teeth structure, a protruded bump structure, a protruded block structure, and a protruded bar structure.

12. The blood pressure meter, as recited in claim 1, wherein each of said first clipping portion and said second clipping portion has at least one hook member, wherein said hook members of said first clipping portion and said second clipping portion are arranged for hooking with said first connecting portion and said second connecting portion respectively such that said first clipping portion and said second clipping portion are locked into position with respective to said first connecting portion and said second connecting portion respectively.

13. The blood pressure meter, as recited in claim 7, wherein each of said first adjustment member and said second adjustment member has at least one hook member, wherein said hook members of said first adjustment member and said second adjustment member are arranged for hooking with said first connecting portion and said second connecting portion respectively such that said first adjustment member and said second adjustment member are locked into position with respective to said first connecting portion and said second connecting portion respectively.

14. A blood pressure meter, comprising:
a cuff unit having a first side and a second side opposite to said first side, comprising at least one first connecting portion and at least one second connecting portion provided on said first side of said cuff unit; and
a clip unit detachably connected with said cuff unit through said first and said second connecting portions on said first side of said cuff unit such that said cuff unit is capable of being open to an open position through said clip unit and being closed to a closed position through said clip unit; wherein said clip unit comprises a first clipping portion corresponding to each said first connecting portion of said cuff unit and a second clipping portion corresponding to each said second connecting portion of said cuff unit, wherein said first clipping portion and said second clipping portion are pivotally connected with each other at a position to define a hinge portion and detachably connected to said first and said second connecting portions respectively in such a manner that said clip unit is capable of being pressed to open said cuff unit and released to close said cuff unit to secure into position through a single-handed operation, thereby a user is capable of being guided to place said first side outwardly through said first and said second connecting portions while positioning said cuff unit and locking said cuff unit into position through said clip unit for measuring blood pressure.

15. The blood pressure meter, as recited in claim 14, further comprising a positioning unit for setting and locking a length of said cuff unit in such a manner that said cuff unit is capable of providing a measuring channel for receiving and enclosing a part of a user for measuring blood pressure, wherein said positioning unit comprises a first position member provided on said cuff unit and a second position member provided on said cuff unit or said clip unit which is arranged for coupling with said first position member in such a manner that said cuff unit is interlocked into position.

16. The blood pressure meter, as recited in claim 15, further comprising a resilient member at said hinge portion of said clip unit arranged for retaining said clip unit at the closed position.

17. The blood pressure meter, as recited in claim 16, wherein said clip unit further comprises a first adjustment member and a second adjustment member complementary and connected to said first clipping portion and said second clipping portion respectively such that a diameter of said measuring channel is adjustable for fitting different users.

18. The blood pressure meter, as recited in claim 17, wherein said first adjustment member and said second adjustment member comprises at least one first locking member provided thereon arranged for further securing said first and second adjustment members into position.

19. The blood pressure meter, as recited in claim 18, wherein said first locking member has a protruded coupling structure for retaining movement of said cuff unit.

20. The blood pressure meter, as recited in claim 19, wherein each of said first clipping portion and said second clipping portion has at least one hook member, wherein said hook members of said first clipping portion and said second clipping portion are arranged for hooking with said first connecting portion and said second connecting portion respectively such that said first clipping portion and said second clipping portion are locked into position with respective to said first connecting portion and said second connecting portion respectively.
